(19) United States
(12) Patent Application Publication
(10) Pub. No.: US 2023/0110187 A1
(43) Pub. Date: Apr. 13, 2023

(54) REINFORCED WORKING CHANNEL TUBE FOR AN ENDOSCOPE

(71) Applicant: AMBU A/S, Ballerup (DK)

(72) Inventors: Kaspar Mat MATTHISON-HANSEN, Helsingør (DK); Michael Kappler HANSEN, Vallensbæk Strand (DK)

(73) Assignee: AMBU A/S, Ballerup (DK)

(21) Appl. No.: 17/964,590

(22) Filed: Oct. 12, 2022

(30) Foreign Application Priority Data
Oct. 13, 2021 (DE) 10 2021 126 571.1

(51) Int. Cl. A61B 1/005

(52) U.S. Cl. CPC A61B 1/0055; A61B 1/0052

(57) ABSTRACT

A method for producing a reinforced working channel tube, a working channel tube produced by the method, a use of the working channel tube and an endoscope including the working channel tube, the method including: providing an elongated hollow tube main body, the tube main body having a meltable plastic material at least on its outer surface; at least locally heating a wire; tensioning the wire and winding the wire at least in sections around the outer surface of the tube main body; softening or melting the outer surface of the tube main body by the wire; securely anchoring or embedding the wire in the outer surface of the tube main body; and forming a coil anchored or embedded in the outer surface of the tube main body through the winding of the wire.

### CROSS REFERENCE TO RELATED APPLICATIONS

This application claims priority from and the benefit of German Patent Application No. 10 2021 126 571.1, filed Oct. 13, 2021, which is incorporated by reference herein in its entirety.

### TECHNICAL FIELD

The present disclosure relates to a reinforced working channel tube for an endoscope, a method for producing the working channel tube, a use of the working channel tube and an endoscope comprising the working channel tube.

### RELATED ART

Known endoscopes usually comprise: an endoscope handle or interface comprising a handle or interface housing and a working channel access port; an insertion cord configured to be inserted into a patient's body cavity and comprising an insertion tube, an actively bendable bending section and a distal tip unit; and an internal working channel extending from the working channel access port of the endoscope handle or interface to the distal tip unit of the insertion cord.

Internal working channels are usually formed by a connector part, the so-called biopsy connector or Y-connector, a flexible working channel tube arranged inside the endoscope handle/interface, the insertion tube and the bending section, and a tip housing of the distal tip unit. A surgical instrument may be guided through the internal working channel into the patient's body cavity, i.e. distally with respect to the tip of the endoscope. Additionally the internal working channel is usually also used as a suction channel to aspirate for example body fluid from an operation area within the patient's body cavity.

In addition to internal working channels there are also already known external working channels which can be mounted to the insertion cord of an endoscope from outside and which may serve as an additional lumen for guiding instruments into the patient's body cavity, for aspirating body fluids from the operation area, etc.

Both internal and external working channels usually comprise a flexible working channel tube having an elongated hollow tube main body usually made of a plastic/polymer material.

When the actively bendable bending section of the insertion cord is bent, e.g. by manually operating a wheel or lever provided at the endoscope handle/interface, there is basically the danger that a kinking of the working channel tube occurs due to its insufficient strength/stability. Kinking usually goes along with a change of the shape of the cross section of the working channel tube. In particular, the cross-sectional shape of the working channel tube is in sections not circular anymore. This may affect a movement of instruments inside the working channel tube and a suction performance.

Therefore, it is basically desirable to reduce or avoid the risk of kinking of the working channel tube during operation of the endoscope, in particular during bending of the actively bendable bending section. It is in this light already known to provide reinforced working channel tubes.

U.S. Pat. No. 6,315,715 discloses a flexible endoscope with a working channel. The working channel is a flexible tube made from a plastic material and has a convoluted portion in which a wall of the tube is folded to increase flexibility. The working channel tube may be manufactured by placing a helical reinforcing coil in its entirety over a portion of the plastic tube and by axially compressing the helical reinforcing coil and said portion of the tube at room temperature or at an elevated temperature, so that said portion of the tube is folded into a convoluted configuration in which the reinforcing coil is secured by convoluted walls of the compressed plastic tube.

Further, U.S. Pat. No. 4,714,075 discloses a working channel for a flexible endoscope, wherein the working channel is a thermoplastic tubular member which can be softened by heat. In order to reinforce the tubular member a cylindrical metal knitted or braided fabric is thermally bonded to the tubular member. The tube and fabric assembly are drawn through a heated die that is maintained at a temperature high enough to soften the outer periphery of the tubular substrate. The metal fabric is forced inwardly into the softened material as it passes through the die cavity which, in turn, displaces substrate material outwardly through the mesh openings whereby the fabric is embedded in the outer surface of the reinforced section.

Moreover, U.S. Pat. No. 4,676,229 discloses an endoscope with a working channel comprising a plastic tube and a metal wire that is helically wound around the plastic tube. The metal wire is bonded to the outer surface of the plastic tube using an adhesive.

In addition, from JP H05-184533 A, JP 4859539 B2 and JP 5189802 B2 it is known to first form or cut a groove on an outer surface of a working channel tube made of plastic material and to afterwards wind an unheated steel wire in the groove so as to form a coil.

It is thus already known from the prior art to provide a reinforced working channel tube. Existing reinforced working channel tubes however inter alia have the problem that their method for producing is expensive and require a plurality of process/ manufacturing steps. Additionally, existing reinforcing methods may not be suitable to reinforce very small working channel tubes with very small wall thicknesses, in particular when the reinforcing materials, e.g. knitted or braided fabrics, may unacceptably reduce the flexibility of the bending section.

### BRIEF DESCRIPTION OF THE DISCLOSURE

In view of the above-described problem, it is an object of the present disclosure to provide a reinforced working channel tube for an endoscope, which can be simply and cost-effectively produced and which effectively reduces the danger of kinking during its operation. It is another object of the present disclosure to provide a reinforced working channel tube in which the working channel tube has a small diameter and/or a thin wall.

These objects are solved by a method for producing a reinforced working channel tube for an endoscope in accordance with claim 1, by a working channel tube in accordance with claim 16, and by an endoscope comprising the working channel tube in accordance with claim 22. Advantageous aspects of the present disclosure are claimed in the dependent claims and/or are described herein below.

The present disclosure relates to a method for producing a reinforced working channel tube for an endoscope, preferably a method for producing a working channel tube, which is reinforced by a coil, the method comprising the following steps: providing an elongated hollow tube main body initially having an essentially smooth inner surface and an essentially smooth outer surface and comprising a meltable plastic material at least on its outer surface; providing a wire, preferably made of metal; at least locally heating the wire; applying a tensioning on the wire and winding the at least locally heated wire at least in sections around the outer surface of the tube main body; softening or melting the outer surface of the tube main body by the wire; securely anchoring or embedding the wire in the outer surface of the tube main body; and forming a coil anchored or embedded in the outer surface of the tube main body through the winding of the wire.

It is to be understood that the reinforced working channel tube produced by said method may be a working channel tube which is entirely reinforced, i.e. over its entire length, or which is only reinforced in sections, over only a portion of its entire length, i.e. only locally.

Moreover, it is to be understood that the essentially smooth inner and outer surfaces of the elongated hollow tube main body relate to inner and outer shell surfaces of the tube main body, which preferably has a round cross-section. Essentially smooth inner and outer surfaces are to be understood as surfaces which have a surface roughness usually obtained in a manufacturing process in which the elongated hollow tube main body is manufactured, e.g. in an extrusion process, preferably without post-processing. Said differently, essentially smooth surfaces are preferably to be understood as unmachined surfaces, which do not comprise grooves, threads, etc.

Further, it is to be understood that the elongated hollow tube main body is preferably a flexible tube. It is to be understood that the feature that at least an outer surface of the elongated hollow tube main body is made of a meltable plastic material means that the elongated hollow tube main body may be entirely made of a meltable plastic material or only/ at least an outer surface of the same may be made of a meltable plastic material. A meltable plastic material is to be understood as plastic/polymer/synthetic material which can melt or soften/which is capable of melting or softening and which preferably has a melting point or range/area. In particular, plastic materials having thermoplastic behavior like thermoplastics, thermoplastic elastomers, etc. are to be understood as meltable plastic materials.

In addition, it is to be understood that “at least locally heating the wire” means that only a local part/portion of the wire may be heated or that the wire may be heated in its entirety.

Furthermore, it is to be understood that “winding the at least locally heated wire at least in sections around the outer surface of the tube main body” means that the wire may be wound around the elongated hollow tube main body only locally, i.e. a portion of the tube main body, or that the wire may be wound around the tube main body along the entire length of the tube main body.

It is to be understood that “embedding the wire in the outer surface of the tube main body” means that the wire may be covered at least in sections along its length or at least around a portion of its circumference in at least some sections, or completely, by the meltable plastic material of the tube main body after the production of the working channel tube. I.e. the melted plastic material may flow over the wire and may close a gap/groove created through the winding of the wire around the tube main body, so that from outside at least in sections or completely the wire is not exposed any more to the environment. The wire may be fixed firmly and deeply in a surrounding mass of material of the tube main body. If the material is not translucent, when the wire is not exposed it is not visible.

Further, it is to be understood that “anchoring the wire in the outer surface of the tube main body” means that the wire may be (alternatively or additionally) in sections or completely only anchored and thus not necessarily be fully/completely covered by the material of the tube main body after the production of the working channel tube. This means that when the wire is anchored in the tube main body it may still visible from outside the tube main body. Preferably, at least 50% of a thickness or diameter of the wire is within the material of the tube main body and beneath the outer surface of the tube main body. While this provides secure anchoring, intermittent partial securement along the wire's length in combination with coiling of the wire may sufficiently affix the wire to the tube main body.

It is thus within the scope of the present disclosure that the wire is covered by the material of the tube main body, i.e. is embedded in the material of the tube main body, i.e. is not visible from outside anymore after production of the working channel tube and that the wire is not covered by the material of the tube main body, i.e. is anchored in the material of the tube main body, i.e. is visible from outside after production of the working channel tube.

In other words, the method for producing a reinforced working channel tube for an endoscope according to the present disclosure may be described such that the (metal) wire/thread is wound around the elongated hollow tube main body and during the winding process, the wire is heated to a specific temperature and is tensioned so that the outer, initially smooth surface of the tube main body melts/softens and the wire cuts into the outer surface and is embedded or anchored in the outer surface/an outer layer of the tube main body. Said differently, during the winding thermal energy/heat is added to the wire in an amount allowing the wire to soften the outer surface of the tube main body made of the meltable plastic material, enabling a secure anchoring or embedding of the wire in the outer surface of the tube main body.

The method for producing a reinforced working channel tube for an endoscope according to the present disclosure provides a number of advantages. As the wire is anchored or embedded in the outer surface of the tube main body, the outer diameter of the tube main body is essentially unchanged or only slightly increased, which is advantageous in particular in the light of the limited available space inside an insertion cord of an endoscope. Moreover, the method preferably does not affect the inner surface of the tube main body, which preferably remains unchanged and smooth, or does only insignificantly affect the inner surface of the tube main body. A small deformation by the coil/small ripples on the inner surface/the inside of the tube main body may be acceptable as long as tools may be advanced through the working channel essentially without increased friction. The method makes it possible that the wire is securely anchored or embedded in the outer surface/outer layer of the tube main body and reinforces the working channel tube. Thus, the wire forms a reinforcing coil and prevents or at least reduces a danger of kinking of the working channel tube. The wire is anchored or embedded in the outer surface/layer of the tube main body during winding without requiring any further production steps. There is no need to heat the tube main body or to push the wire into the outer surface of the tube main body afterwards. The wire is inserted/embedded/anchored into the tube main body from the outside essentially through the winding tension and its temperature and it is not necessary according to the present disclosure to provide grooves in the outer surface of the tube main body before winding the wire around the same. The winding is thus preferably performed directly on the smooth outer surface of the tube main body. The method according to the present disclosure is thus simple, cost-effective and adaptable to specific purposes. Compared to prior art methods for producing reinforced working channel tubes several complicated process steps may be eliminated.

The method preferably comprises the step: using the working channel tube having the securely anchored or embedded wire in the outer surface of the tube main body, thereby forming the coil, as part of an internal or external working channel of an endoscope.

Preferably, the method further comprises the step: creating a specific or desired pitch of the coil, preferably between 0.5 mm and 1.5 mm, or between 0.8 mm and 1.2 mm, or about 1.0 mm. Especially preferred the method comprises the steps: turning the tube main body around its longitudinal axis; axially moving the tensioned wire (in particular a wire providing device like a winch) along the longitudinal axis of the tube main body; and creating a specific or desired pitch of the coil, preferably between 0.5 mm and 1.5 mm, especially preferred about 1.0 mm, by correspondingly setting a (rotational) speed of the tube main body and an axial translational velocity of the tensioned wire.

Turning the tube main body around its longitudinal axis may allow winding the wire around the tube main body without having to move the wire in a circumferential direction. A spindle may for example turn the tube main body. By controlling the axial movement of the wire/the wire providing device, the pitch of the coil may be set or adjusted as requested. The wire may move in the axial direction and may be stationary in a circumferential direction of the tube main body. This may lead to a cost-effective production of the reinforced working channel tube. For example, a winch may be provided to wind the wire around the tube main body. By appropriately adjusting/setting the pitch a reinforcement-degree of the working channel tube may be set. It is noted that the reinforcement-degree of the tube main body may alternatively or additionally be adjusted by changing a cross-sectional shape or area of the wire.

It is to be understood that the present disclosure is not limited to the embodiment according to which the tube main body is turned around its longitudinal axis and the tensioned wire is axially moved along the longitudinal axis of the tube main body. The tube main body e.g. may also be stationary/fixed and the wire may be moved both axially and circumferentially with respect to the longitudinal axis of the tube main body. Further alternatively, the wire may be held stationary, i.e. may not move axially if the tube main body is both rotated and axially moved. Also with these alternative embodiments, a specific or desired pitch of the coil may be appropriately set.

Preferably, the method further comprises the step: heating the wire only locally immediately before winding the locally heated wire around the smooth outer surface of the tube main body. When the wire is heated only locally immediately before winding the same around the tube main body it may be ensured that occurring heat losses are reduced/minimized and an energy-efficient production/manufacturing method is provided. Said differently, it is preferred to heat the wire only at a portion of the same, which is immediately afterwards wound around the tube main body, so that said portion may cut into and melt the outer surface/layer of the tube main body easily and effectively. The wire may e.g. be heated by contact heating, preferably by a heating element. Contact heating is preferred according to the present disclosure as it may produce less heat losses compared to contactless heating. The use of the heating element is e.g. a cost-effective way to heat the wire. However, it is to be understood that any heating method/means may be used/provided as long as it enables the wire to be locally or entirely heated.

Preferably, the method further comprises the step: tensioning the wire between 0.1 N and 10 N, preferably between 0.5 N and 3 N, or between 1.6 N and 2.4 N or about 2 N, during the winding. This may ensure—together with the heating—that the wire may be securely anchored or embedded in the outer surface/layer of the tube main body. The tensioning (force) may be set such that the wire does not cut entirely through the tube main body. Controlling the tensioning of the wire may thus make it possible to control how deep the wire cuts into the outer surface of the tube main body. It is to be understood that the tensioning force applied is of course dependent on properties/materials of the tube main body and the wire and other tensioning forces are basically conceivable.

Preferably, the method further comprises the step: winding the wire only in sections around the outer surface of the tube main body, i.e. not along an entire length of the tube main body, thus providing an only locally reinforced working channel tube. The properties of the reinforced working channel tube may be varied by reinforcing only sections of the working channel tube. The wire may e.g. be added only in a local area, for example an area of the working channel tube which is arranged in/inside an actively bendable bending section of an insertion cord of an endoscope. Thereby the coil formed by the wound wire may locally improve the kinking resistance and may ensure that a circular cross section of the working channel tube is kept during bending. By providing local reinforcement a working channel tube having varying properties along its length may be produced. As the reinforcement according to the present disclosure preferably does not affect or does only insignificantly affect the smooth inner surface of the tube main body, movement behavior of surgical instruments through the working channel and suction performance are not impaired by providing only local reinforcement of the working channel tube.

Preferably, the method further comprises the step: providing the elongated hollow tube main body with an outer layer comprising the outer surface and an inner layer comprising the inner surface, wherein a material of the outer layer is different from a material of the inner layer. When the two layers, i.e. the outer layer and the inner layer, are made of different material, each layer may be optimized for its specific purpose. The outer layer of the tube main body is made of a meltable plastic material that is configured to melt or soften when it is heated according to the present disclosure, thus allowing the hot wire to melt or soften the outer layer and cut into the same when it is wound around the tube main body. The inner layer may be made of a meltable plastic material, however does not have to be made of the same, as it is not intended to be melted or softened. The inner layer and thus its material may be optimized for other purposes, e.g. enabling a smooth gliding of an instrument inside the working channel tube. The outer layer may e.g. be made of TPU (thermoplastic polyurethane) and the inner layer may be made of HDPE (high-density polyethylene).

According to a preferred embodiment, the materials of the outer layer and the inner layer may both be meltable plastic material and a melting temperature or range/area of the material of the inner layer may be higher than a melting temperature or range/area of the material of the outer layer. This makes it possible to adjust a temperature of the heated wire such that said temperature is above the melting temperature/range/area of the material of the outer layer and below the melting temperature/range/area of the material of the inner layer. This preferred embodiment makes it possible that the wire cannot melt or soften the inner layer, as its temperature is not high enough. A cut-in deepness of the wire may thus be appropriately defined, essentially independently from the tensioning force applied to the wire.

The inner layer may be thinner than the outer layer, or said differently, a wall thickness of the inner layer may be smaller than a wall thickness of the outer layer. E.g. the inner layer may have a thickness of between 0.050 mm to 0.080 mm and the outer layer may have a thickness of between 0.32 mm to 0.35 mm. However, other layer thicknesses are conceivable and it is also conceivable that the thickness of the inner layer is greater than the thickness of the outer layer. The tube main body wall may have a wall thickness of less than 0.5 mm, or less than 0.4 mm.

The reinforced tube main body wall may have a wall thickness of less than 0.5 mm, or less than 0.4 mm, and may comprise an inner layer having a thickness of between 0.050 mm to 0.080 mm, an outer layer having a thickness of between 0.32 mm to 0.35 mm, and a coil with a coil pitch between 0.5 mm and 1.5 mm, or between 0.8 mm and 1.2 mm, or about 1.0 mm. The diameter/thickness of the wire may be between 0.08 mm and 0.15 mm. The wall thickness of the tube main body is, generally, at least two times greater than the diameter or thickness of the wire.

Optionally, a primer may be provided between the inner layer and the outer layer to ensure and increase an adherence between the two layers. This is e.g. of advantage if the outer layer is made of TPU (thermoplastic polyurethane) and the inner layer is made of HDPE (high-density polyethylene). The tube main body may be coextruded, thus the inner, tie or primer, and outer layers are extruded simultaneously.

According to an alternative preferred embodiment the tube main body may also be an integral part made of only one meltable plastic material, thus not comprising the outer layer and inner layer made of different materials. In this case the tensioning force and the temperature of the wire should be suitable set (e.g. by performing experiments) such that the wire does not cut through the entire wall thickness of the tube main body.

The method preferably comprises the step: fixing an end of the wire to the tube main body after forming the coil.

Preferably, the wire is broken off after completion of the winding, e.g. by bending the wire multiple times in different directions. It may be especially preferred if a sharp bend, in particular a kink, is provided to the wire at the outer surface of the tube main body before bending the wire back and forth multiple times in different directions. E.g. the end of the wire may be pushed against the tube main body, in particular against the outer surface of the tube main body, at a specific position, and the sharp bend of the wire may be provided at said specific position. The sharp bend is preferably performed such that a weakness point or starting point for breaking off the wire is provided on the outer surface of the tube main body or at least very close to the outer surface of the tube main body so that the wire is preferably broken flush with the outer surface of the tube main body when the wire is subsequently bent back and forth multiple times in different directions. The sharp bend can e.g. be manually provided by pushing the wire against the specific position by a fingernail, or automatically by a pushing or pressing body. Breaking the wire flush with the outer surface of the tube main body provides the advantage that a risk of a sharp wire end sticking out of the outer surface of the tube main body is reduced, thereby reducing a potential risk of damaging other components of the endoscope, such as a camera wire, other tubes, etc., in particular in the assembly process. Alternatively, the wire may be cut after termination of the winding process.

The risk of the wire end sticking out of the outer surface of the tube main body may be further reduced, in particular after breaking off the wire by the sharp bend as mentioned above, by adding a drop of adhesive to the wire end, thereby covering the wire end with adhesive, and fixing the wire end to the tube main body. The end of the wire may be additionally or alternatively anchored or embedded in the tube main body after the winding is completed, e.g. by locally heating and melting the tube main body and pressing the end of the wire into the locally heated and melted portion of the tube main body. Thus, the end of the wire may be fixed to the tube main body. Said differently, the method preferably also comprises the step: fixing a free end of the wire to the tube main body by locally heating and melting a portion of the tube main body and pressing the free end of the wire into said portion of the tube main body. Additionally or alternatively, fixing a free end of the wire to the tube main body may comprise locally heating the free end of the wire and then pressing the free end to melt a portion of the tube main body into said portion of the tube main body.

The present disclosure may also relate to a method for producing an endoscope comprising the method for producing the reinforced working channel tube as described above and a step of introducing/assembling the reinforced working channel tube having the securely anchored or embedded wire in the outer surface of the tube main body, thereby forming the coil, into an endoscope, e.g. as part of an internal or external working channel.

The present disclosure also relates to a working channel tube comprising: an elongated hollow tube main body, preferably having a round cross section, having an essentially smooth inner surface and comprising a meltable plastic material at least on its outer surface; and a coil, preferably made of metal, firmly attached and anchored or embedded in the outer surface of the tube main body, the coil being formed by: providing a wire; at least locally heating the wire; applying a tensioning on the wire and winding the at least locally heated wire at least in sections around an initially smooth outer surface of the tube main body; softening or melting the outer surface of the tube main body by the wire; and securely anchoring or embedding the wire in the outer surface of the tube main body.

Expressed in other words, the working channel tube comprises the elongated hollow tube main body and the (metal) reinforcing coil anchored or embedded in the outer surface of the tube main body. The reinforcing coil is formed by winding the (metal) wire around the tube main body. During the winding, thermal energy/heat is added to the wire in an amount allowing the wire to soften the outer surface of the tube main body made of the meltable plastic material, enabling a secure anchoring or embedding of the wire in the outer surface of the tube main body. The working channel tube according to the present disclosure is thus manufactured applying a specific process having a small number of production steps and being cost-effective. Said process makes it possible to at least locally/in sections reinforce the working channel tube, thus making the reinforced working channel tube less vulnerable to kinking. The working channel tube may e.g. be reinforced in an area, which is intended to be arranged in an actively bendable bending section of an endoscope.

The working channel tube having the coil firmly attached and anchored or embedded in the outer surface of the tube main body is preferably (sterile) packaged in a (sterile) packaging, i.e. the working channel tube is preferably a (sterile) packaged working channel tube. This has the advantage that the working channel tube may be independently manufactured and stored, and may be introduced/assembled into an endoscope as part of an internal or external working channel anytime thereafter.

Preferably, the inner diameter of the working channel tube is greater than 2 mm and smaller than 4.5 mm. Kinking is a problem that may depend on the inner diameter of the working channel tube, and it has been found that tubes having an inner diameter of 2 mm or less may not have the problem of kinking. However, it is nevertheless conceivable to also reinforce such small-diameter working channel tubes having an inner diameter of less than 2 mm. Because of a limited outer diameter of an insertion cord of the endoscope and a limited space inside the insertion cord, the inner diameter of the tube main body is preferably less than 4.5 mm. A preferred inner diameter of the tube main body is around 3.8 mm according to the present disclosure. A diameter smaller than 3.0 mm is also envisioned, for use with an insertion tube having an external diameter of 3.4 mm or less.

Preferably, a pitch of the coil is between 0.5 mm and 1.5 mm, especially preferred around 1 mm.

Preferably, the elongated hollow tube main body comprises an outer layer comprising the outer surface and an inner layer comprising the inner surface, wherein a material of the outer layer is different from a material of the inner layer. Especially preferred, the materials of the outer layer and of the inner layer are both meltable plastic materials and a melting temperature of the material of the inner layer is higher than a melting temperature of the material of the outer layer.

Preferably, the outer diameter of the reinforced working channel tube is essentially the same as the outer diameter of the elongated hollow tube main body. As the wire is preferably completely anchored or embedded in the outer surface of the tube main body, the wire preferably “disappears” in the tube main body, i.e. preferably does not protrude radially from the outer surface of the tube main body. The outer diameter of the reinforced tube main body is thus preferably not increased or only increased by a very small amount compared to the initial tube main body. This may be advantageous when the reinforced working channel tube is arranged inside an insertion cord of an endoscope, where only limited space is available.

Preferably, the coil/the wire is made of metal. Especially preferred, the coil/wire is made of steel, e.g. stainless steel. However, any other metal materials like copper, titanium, etc. are also conceivable. Moreover, also non-metal materials are conceivable for the coil/the wire as long as the wire made of said material can be appropriately heated and tensioned.

The wire used to form the coil preferably has a round cross-section, however flat wires may basically also be used.

Preferably, the wall thickness of the tube main body is at least two times greater than a diameter or thickness (in case of a flat wire) of the wire, so that the wire can be suitably anchored or embedded in the outer surface of the tube main body. E.g., the wall thickness of the tube main body may be between 0.3 mm and 0.5 mm, e.g. around 0.4 mm and the diameter/thickness of the wire may be between 0.08 mm and 0.15 mm. Other wall thicknesses and wire diameters/thicknesses may be used as long as the wall thickness of the tube main body is at least two times greater than the diameter or thickness of the wire.

The present disclosure also relates to a use of a working channel tube as described above as part of an internal or external working channel of an endoscope.

Further, the present disclosure relates to an endoscope comprising: an endoscope handle or interface comprising a handle or interface housing and a working channel access port; an insertion cord configured to be inserted into a patient's body cavity and comprising an insertion tube, an actively bendable bending section and a distal tip unit; and a working channel extending from the working channel access port of the endoscope handle or interface to the distal tip unit of the insertion cord and comprising a working channel tube as described above.

Preferably, the working channel tube extends through the bending section and is locally reinforced by the coil along a length of the working channel tube arranged in the bending section.

### BRIEF DESCRIPTION OF FIGURES

The disclosure is explained in more detail below using preferred embodiments and referring to the accompanying figures.

FIG. 1 shows a schematic view of an embodiment of an endoscope according to the present disclosure.

FIG. 2 shows a flow chart of a method for producing a working channel tube according to the present disclosure.

FIG. 3 shows the working channel tube according to the present disclosure during its production.

FIG. 4 shows an arrangement of a tube main body in a winding apparatus.

FIG. 5 shows a cross-section through a tube main body according to an embodiment of the present disclosure.

FIG. 6 is a longitudinal sectional view of a working channel tube having a coil embedded in a tube main body according to an embodiment of the present disclosure.

FIG. 7 is a longitudinal sectional view of a working channel tube having a coil anchored in a tube main body according to an embodiment of the present disclosure.

FIG. 8 is a side view of a bending section showing a working channel tube.

FIG. 9 is cross-section A-A of the bending section of FIG. 8.

FIG. 10 is a side view of a visualization system including another embodiment of an endoscope according to the present disclosure.

The figures are schematic in nature and serve only to understand the disclosure. The features of the different embodiments can be interchanged among each other.

### DETAILED DESCRIPTION

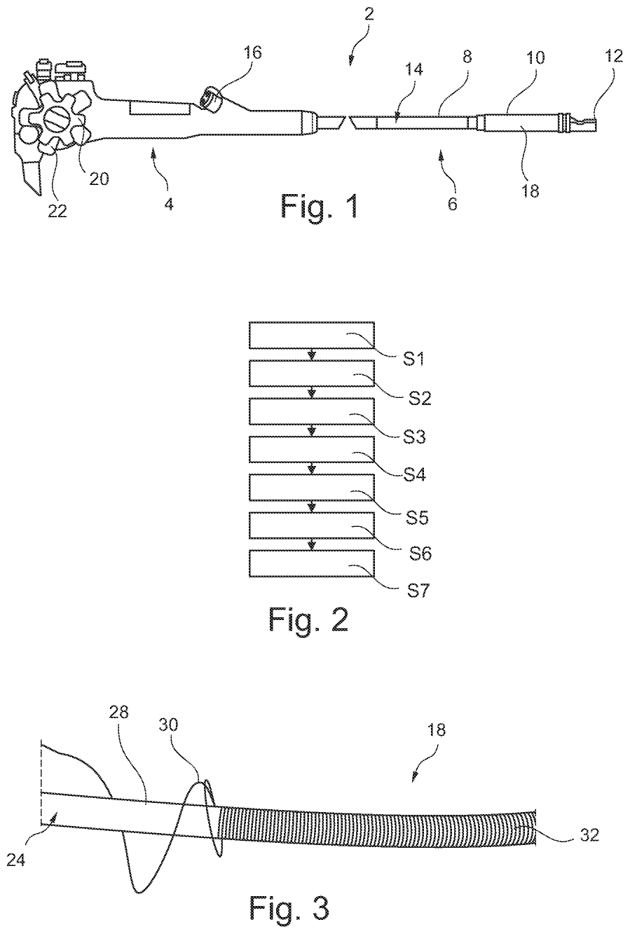

FIG. 1 shows a schematic view of an endoscope 2, which is preferably a single-use endoscope. The endoscope 2 has a proximal endoscope handle 4 and an insertion cord 6 extending distally from the endoscope handle 4. The insertion cord 6 is configured to be inserted into a patient's body cavity and comprises an insertion tube 8, an actively bendable bending section 10 and a distal tip unit 12. The endoscope 2 further comprises a working channel 14 which extends from a working channel access port 16 provided at the endoscope handle 4 to the distal tip unit 12. The working channel 14 is comprised by a working channel tube 18, which is arranged inside the endoscope handle 4, the insertion tube 8 and the bending section 10. The endoscope handle 4 comprises two operating units 20, 22 formed as wheels for steering the bending section 10 of the insertion cord 6. In particular, a rotation/turning force can be applied to both the first operating unit 20 and the second operating unit 22 by a user in order to bend the bending section 10 in two bending planes/four directions. The endoscope 2 may alternatively be formed as a one-plane bending endoscope which can be bent only in two, preferably opposite, directions and which has only one operating unit 20, 22. In addition to wheels, operating units may comprise levers as are well known in the art, an example of which is shown in FIG. 10.

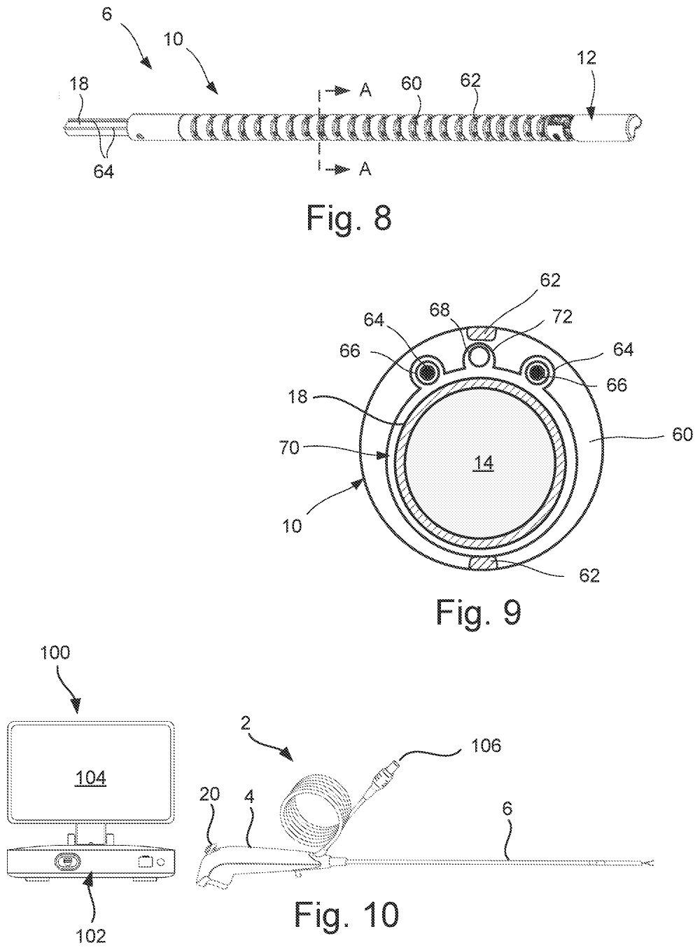

FIG. 8 shows an embodiment of a bending section 10 showing a working channel tube 18 (the reinforcement is not shown). FIG. 9 is cross-section A-A of the bending section of FIG. 8 showing a spacing 70 in which the working channel tube 18 is positioned. The bending section 10 may comprise a single-piece polymeric structure comprising a plurality of segments 60 interconnected by polymeric strips 62, or hinges, which form part of the one-piece structure and bend upon tensioning of steering cables 64 by operation of the operating unit 20. The working channel 14 may be configured to introduce a surgical tool therethrough. In FIG. 9, a wall of the segment 60 defines the spacing 70 and a number of cut-out lobes 72 extending from the periphery of the inner surface of the segment 60 through which steering cable guide tubes 66 and the steering cables 64 pass. A sleeve (not shown) may be provided over the bending section 10 to fluidly seal the spaces between adjacent segments 60. Wires 68 pass through one of the cut-out lobes 72 and connect a camera positioned in the distal tip of the insertion cord 6 to the handle 4. A cable having a cable connector 106 connects the wires 68 to a video processing apparatus (VPA) 102 shown in FIG. 10. As can be envisioned, space in the bending section is limited and becomes even more limited as the diameter of the insertion cord 6 is reduced to minimize the invasiveness of the surgical procedure in which the endoscope 2 is used. These reductions require smaller working channel tubes with smaller wall thicknesses, and the working channel tubes with reduced wall thickness benefit from the disclosed reinforcement.

FIG. 10 is a side view of a visualization system 100 including another embodiment of an endoscope 2 showing the cable connector 106, which can be connected to an endoscope connection port (not shown) of the VPA 102. A VPA can incorporate a display screen 104 or, as shown, can be connected to a “separable” display screen. The VPA can be communicatively connected, as is known in the art, via an ethernet, wireless, AVI, HDMI, or other data interfaces to a separable display screen 104. Upon connection of the endoscope the VPA presents images or video streams with the display screen, as is known in the art.

The working channel tube 18 is a specific reinforced working channel tube 18 according to the present disclosure and is in particular reinforced in a portion of the working channel tube 18 which is arranged inside of the bending section 10, in order to prevent kinking of the working channel tube 18 when the bending section 10 is bent. It is to be understood that it is also conceivable to reinforce the entire working channel tube 18, i.e. not only the portion of the working channel tube 18 which is arranged inside the bending section 10 of the insertion cord 6, or other portions of the working channel tube 18. Further, it is to be understood that the working channel tube 18 does not need to be arranged inside the insertion cord 6 but may alternatively also be attached to the insertion cord 6 from outside.

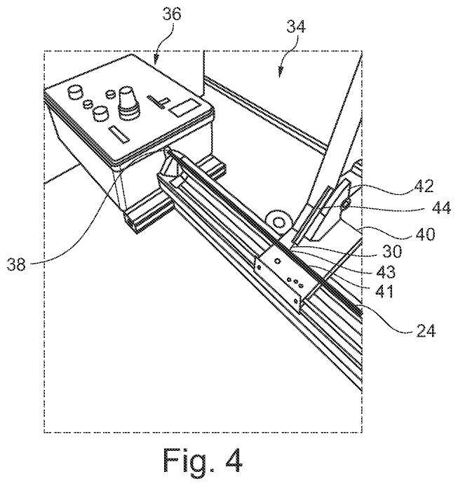

With reference to FIG. 2, FIG. 3 and FIG. 4 a method for producing a reinforced working channel tube 18 according to the present disclosure is described. FIG. 2 shows a flow chart of the method for producing a reinforced working channel tube 18. In a first step S1, an elongated hollow tube main body 24 comprising an inner surface 26 and an outer surface 28 is provided. The tube main body 24 comprises, at least on its outer surface 28, a meltable plastic material. The inner surface 26 and the outer surface 28 may, initially, when provided, be essentially smooth, e.g. devoid of grooves, channels, and/or corrugations.

In a step S2 a wire 30, preferably made of metal, is provided. The wire 30 is at least locally heated in a step S3.

In a step S4 a tensioning is applied on the wire 30 and the at least locally heated wire 30 is at least in sections directly wound around the outer surface 28 of the tube main body 24. The heated wire 30 softens or melts the outer surface 28 of the tube main body 24 in a step S5. In a further step S6 the wire is securely anchored or embedded in the outer surface 28 of the tube main body 24. In a step S7 a coil 32 anchored or embedded in the outer surface 28 of the tube main body 24 is formed through the winding of the wire 30. It is to be understood that the process steps S4 to S7 are performed simultaneously/follow immediately one after another. In particular, when the at least locally heated wire 30 is wound around the outer surface 28 of the tube main body 24, it is a direct consequence of the temperature of the wire 30 and the tension applied on the wire 30 that the wire 30 softens/melts the outer surface 28 of the tube main body 24. Moreover, a direct consequence of the softening/melting of the outer surface 28 of the tube main body is the secure anchoring/embedding of the wire 30 in the outer surface 28 of the tube main body 24. The formation of the coil 32 is the direct consequence of the winding and anchoring/embedding.

The method preferably comprises fixing a first end of the wire to the tube main body before forming the coil. Fixing the end of the wire to the tube main body facilitates tensioning and coiling of the wire around the tube main body. The wire may be broken, from a spool or portion of wire that is not attached, flush with the outer surface of the tube main body. This provides the advantage that a risk of a sharp wire end sticking out of the outer surface of the tube main body is reduced. Fixing the end of the wire may comprise heating the end of the wire and applying pressure onto the end of the wire to locally melt the tube main body and thus, upon cooling, securing the end of the wire to the tube main body. The end of the wire may be bent at a sharp angle to form small hook and thus prevent the tip of the end of the wire to potentially damage other parts of the endoscope.

The method preferably comprises fixing a second end of the wire, opposite the first end, to the tube main body after forming the coil. Fixing the end of the wire may comprise heating the end of the wire and applying pressure onto the end of the wire to locally melt the tube main body and thus, upon cooling, securing the end of the wire to the tube main body. The end of the wire may be bent at a sharp angle to form small hook and thus prevent the tip of the end of the wire to potentially damage other parts of the endoscope. The wire may be broken flush with the outer surface of the tube main body. This provides the advantage that a risk of a sharp wire end sticking out of the outer surface of the tube main body is reduced.

Preferably, the wire is broken off after completion of the winding, e.g. by bending the wire multiple times in different directions. It may be especially preferred if a sharp bend, in particular a kink, is provided to the wire at the outer surface of the tube main body before bending the wire back and forth multiple times in different directions. E.g. the end of the wire may be pushed against the tube main body, in particular against the outer surface of the tube main body, at a specific position, and the sharp bend of the wire may be provided at said specific position. The sharp bend is preferably performed such that a weakness point or starting point for breaking off the wire is provided on the outer surface of the tube main body or at least very close to the outer surface of the tube main body so that the wire is preferably broken flush with the outer surface of the tube main body when the wire is subsequently bent back and forth multiple times in different directions. The sharp bend can e.g. be manually provided by pushing the wire against the specific position by a fingernail, or automatically by a pushing or pressing body. Breaking the wire flush with the outer surface of the tube main body provides the advantage that a risk of a sharp wire end sticking out of the outer surface of the tube main body is reduced, thereby reducing a potential risk of damaging other components of the endoscope, such as a camera wire, other tubes, etc., in particular in the assembly process. Alternatively, the wire may be cut after termination of the winding process.

The risk of the wire end sticking out of the outer surface of the tube main body may be further reduced, in particular after breaking off the wire by the sharp bend as mentioned above, by adding a drop of adhesive to the wire end, thereby covering the wire end with adhesive, and fixing the wire end to the tube main body. The end of the wire may be additionally or alternatively anchored or embedded in the tube main body after the winding is completed, e.g. by locally heating and melting the tube main body and pressing the end of the wire into the locally heated and melted portion of the tube main body. Thus, the end of the wire may be fixed to the tube main body. Said differently, the method preferably also comprises the step: fixing a free end of the wire to the tube main body by locally heating and melting a portion of the tube main body and pressing the free end of the wire into said portion of the tube main body. Additionally or alternatively, fixing a free end of the wire to the tube main body may comprise locally heating the free end of the wire and then pressing the free end to melt a portion of the tube main body into said portion of the tube main body.

FIG. 3 shows the working channel tube 18 for the endoscope 2 during production. The working channel tube 18 comprises the tube main body 24 and the wire 30. The wire 30 is wound around the tube main body 24 and during the winding process, the wire 30 is locally heated to a specific temperature and is tensioned, e.g. by a tensioning force of around 2 N, so that the outer surface 28 of the tube main body 24 melts and the wire 30 cuts into and is embedded/anchored in the outer surface 28 of the tube main body 24. The wire 30 is preferably a metal wire, e.g. a steel thread, in particular stainless steel. Moreover, the wire preferably has a round cross-section and a wire diameter of between 0.15 and 0.07 mm, between 0.12 and 0.08 mm, e.g. 0.10 mm. The wire diameter may be selected based on the wall thickness and the composition of the tube main body 24. The wound wire 30 forms a reinforcing coil 32 at least in sections around the tube main body 24. The winding of the wire 30 is performed such that a specific desired pitch may be reached, which serves to adjust a reinforcement-degree. E.g. the pitch can be set such that it is between 0.5 mm and 1.5 mm, e.g. around 1 mm. The reinforcement-degree may additionally or alternatively be adjusted by appropriately setting a cross-sectional area of the wire 30. The tube main body 24 may have an inner diameter (defined by the inner surface 26) of around 3.8 mm or less, 3.2 mm or less, or 2.8 mm or less. A wall thickness of the tube main body 24 may equal to or less than 0.5 mm, or 0.4 mm or less.

FIG. 4 shows an arrangement of the tube main body 24 in a winding apparatus 34. The winding apparatus 34 comprises a control unit 36, a spindle 38, a slide 40, a winch 42 and a heating element 44. The heating element 44 comprises a metal portion and an electrical resistive element capable of generating heat upon the conduction therethrough of electrical current, e.g. a soldering iron. The tube main body 24 is mounted to the spindle 38 and may be rotated by the spindle 38. The winch 42 and the heating element 44 are fixedly connected to the slide 40, which may be displaced/moved in an axial direction of the tube main body 24. The slide may comprise a slot 41 over which the tube main body 24 rotates. The slot 41 depth can be used to ensure the wire 30 approaches the outer surface 28 of the tube main body 24 at a relative position to ensure the wire 30 does not penetrate too deeply into the wall 25 of the tube main body 24. The slot 41 may also provide a measure of cooling after the heated wire is wound. A guide surface 43 on the slide can be used instead to set the approach angle and position of the wire. The control unit 36 controls parameters of the production process, e.g. the axial translation velocity of the slide 40 or the rotational speed of the spindle 38 or a tensioning force applied on the wire 30. The winch 42 may provide the wire 30. The wire 30 is locally heated by passing the wire 30 over the metal surface of the heating element 44 and is then directly wound around the tube main body 24. The winding is performed by rotating the tube main body 24 around its longitudinal axis by the spindle 38 and by axially moving the slide 40 and the winch 42 (and thus the wire 30) along the longitudinal axis of the tube main body 24 according to this preferred embodiment. A pair of opposing wheels, with the wire 30 passing therebetween, may apply a braking force to set the tension on the wire 30 in combination with the rotating speed of the spindle.

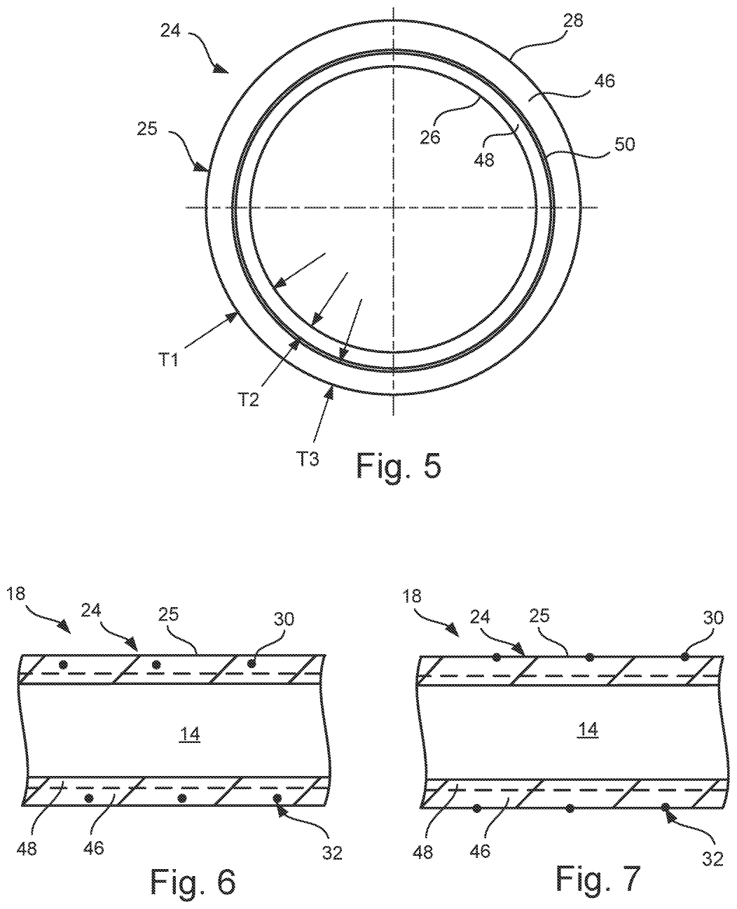

FIG. 5 shows a cross-section through the tube main body 24 according to an embodiment of the present disclosure. The tube main body 24 has a circular cross-section and comprises two layers, namely an outer layer 46 and an inner layer 48. Both the outer layer 46 and the inner layer 48 are essentially hollow elongated tube bodies and are preferably made of different materials. The outer layer 46 is provided directly around the inner layer 48, or said differently the inner layer 48 is inserted into the outer layer 46. A primer 50 may be provided between the outer layer 46 and the inner layer 48 in order to ensure adherence between the outer layer 46 and the inner layer 48. E.g. the outer layer 46 may be made of thermoplastic polyurethane (TPU) and the inner layer 48 may be made of high-density polyethylene (HDPE). The layers may be coextruded. T1 represents the wall thickness. T2 represents the inner layer thickness. T3 represents the outer layer thickness.

The materials used for the outer layer 46 and the inner layer 48 may also be chosen such that they are both meltable plastic materials and a melting temperature of the material of the inner layer 48 is higher than a melting temperature of the material of the outer layer 46. In this case, the temperature of the wire 30 can be adjusted such that it is between the melting temperature of the material of the outer layer 46 and the melting temperature of the material of the inner layer 48. The melting temperature of a polymer is a temperature at which a crystalline polymer converts to a liquid, or crystalline domains of a semi-crystalline polymer melt. Pellethane® 2363-90AE, for example, has an extrusion melt temperature of 370-400° F., and a Vicat Softening Point of 165° F. (ASTM D1525 2° C./min, 9,8N). By using a material in the inner layer that softens at a higher temperature than the material of the outer layer it is possible to increase the wire temperature range without damaging the integrity of the tube main body, regardless which temperature measurement technique is used.

The embodiment shown in FIG. 5 is only an example and it is evident that it may be also advantageous if the tube main body 24 is not made of two parts (the outer layer 46 and the inner layer 48) but is made of one integral elongated tube main body part having the outer surface 28 and the inner surface 26 and being made of a meltable plastic material.

Embodiments according to which the tube main body 24 is made of one integral part are shown in FIG. 6 or in FIG. 7. FIG. 6 shows a longitudinal sectional view of a working channel tube 18 having a coil 32 embedded in a tube main body 24. The wire 30 forming the coil 32 is covered by a material of the tube main body 24 and is thus not exposed to (visible from) the outside, since the melted plastic material has flown over the wire 30/has closed a gap or groove created through the winding of the wire 30 around the tube main body 24 during the production process. FIG. 7 shows a longitudinal sectional view of a working channel tube 18 having a coil 32 anchored in a tube main body 24. The wire 30 forming the coil 32 is not entirely covered by the material of the tube main body 24 after the production of the working channel tube 18 and is thus still visible from outside. Preferably, more than 50% of the diameter, measured perpendicular to the outer surface, is embedded in the material, such that upon cooling the material mechanically affixes the wire.

According to an especially preferred embodiment, the tube main body 24 may have an outer diameter of 5.4±0.076 mm, an inner diameter of 4.4±0.076 mm and a tube wall thickness of 0.5 mm. The total length of the tube main body 24 may be 1,500 mm according to the especially preferred embodiment. The tube main body 24 may comprise an outer layer 46, an inner layer 48 and a primer 50, which can also be designated as tie layer, between the outer layer 46 and the inner layer 48 according to the especially preferred embodiment. The material of the outer layer 46 may be a thermoplastic polyurethane like Pellethane® 2363-90AE with additives according to the especially preferred embodiment. E.g., the outer layer 46 may comprise 70% Pellethane® 2363-90AE and 30% additives. Among additives, it may be preferred if the outer layer 46 comprises an antimicrobial additive like BaSo4 and a friction reducing additive like ProPell®. The material of the inner layer 48 may be a high-density polyethylene like Borealis Bormed® HE9621-PH. The material of the primer 50 or tie layer may be a linear low-density polyethylene based tie resin like Orevac® 18300M.

The wire 30 may have an outer diameter of 0.15 mm according to the especially preferred embodiment. The material of the wire may be stainless steel according to the especially preferred embodiment, e.g. SUS304. The wire made of SUS304 may comprise a Br—Ni—Co coating.

The pitch of the coil 32 formed through the winding of the wire 30 may be 1 mm according to the especially preferred embodiment. The length of the section of the tube main body 24, which is reinforced by the coil 32, may be 115±5 mm according to the especially preferred embodiment. The section reinforced by the coil 32 may have an inner diameter, which is greater than 4.2 mm, and an outer diameter, which is smaller than 5.4 mm.

The kinking performance of the tube main body 24 according to the especially preferred embodiment with and without reinforcement by the coil 32 has been tested. In particular, bending diameters of 23 mm, 30 mm, 40 mm, 50 mm and 60 mm have been tested. It has shown that the tube main body 24 without reinforcement by the coil 32 does kink for bending diameters of 23 mm, 30 mm and 40 mm and does not kink for bending diameters of 50 mm and 60 mm. The tube main body 24 with reinforcement by the coil 32 does only kink for a bending diameter of 23 mm and does not kink for bending diameters of 30 mm, 40 mm, 50 mm and 60 mm. Therefore, it has shown that according to the present disclosure kinking performance of the working channel tube 18 may be improved.

The following items are examples of various embodiments and variations thereof disclosed above and others:

Item 1. A method for producing a reinforced working channel tube (18) for an endoscope (2), the method comprising: providing an elongated hollow tube main body (24) initially having an essentially smooth inner surface (26) and an essentially smooth outer surface (28) and comprising a meltable plastic material at least on its outer surface (28); providing a wire (30), preferably made of metal; at least locally heating the wire (30); applying a tensioning on the wire (30) and winding the at least locally heated wire (30) at least in sections around the outer surface (28) of the tube main body (24); softening or melting the outer surface (28) of the tube main body (24) by the wire (30); securely anchoring or embedding the wire (30) in the outer surface (28) of the tube main body (24); and forming a coil (32) anchored or embedded in the outer surface (28) of the tube main body (24) through the winding of the wire (30).

Item 2. The method according to item 1, further comprising: turning the tube main body (24) around its longitudinal axis; axially moving the tensioned wire (30) along the longitudinal axis of the tube main body (24); and creating a specific or desired pitch of the coil (32), preferably between 0.5 mm and 1.5 mm, especially preferred around 1 mm, by correspondingly setting a rotational speed of the tube main body (24) and an axial movement velocity of the wire (30).

Item 3. The method according to item 1 or 2, further comprising: heating the wire (30) only locally immediately before winding the locally heated wire (30) around the smooth outer surface (28) of the tube main body (24).

Item 4. The method according to any one of items 1 to 3, further comprising: tensioning the wire (30) between 0.1 N and 10 N, preferably between 0.5 N and 3 N, especially preferred around 2 N, during the winding.

Item 5. The method according to any one of items 1 to 4, further comprising: winding the wire (30) only in sections around the outer surface (28) of the tube main body (24), i.e. not along an entire length of the tube main body (24), thus providing an only locally reinforced working channel tube (18).

Item 6. The method according to any one of items 1 to 5, further comprising: providing the elongated hollow tube main body (24) with an outer layer (46) comprising the outer surface (28) and an inner layer (48) comprising the inner surface (26), wherein a material of the outer layer (46) is different from a material of the inner layer (48).

Item 7. A working channel tube (18) comprising: an elongated hollow tube main body (24) having an essentially smooth inner surface (26) and comprising a meltable plastic material at least on its outer surface (28); and a coil (32), preferably made of metal, firmly attached and anchored or embedded in the outer surface (28) of the tube main body (24), the coil (32) being formed by: providing a wire (30); at least locally heating the wire (30); applying a tensioning on the wire (30) and winding the at least locally heated wire (30) at least in sections around an initially smooth outer surface (28) of the tube main body (24); softening or melting the outer surface (28) of the tube main body (24) by the wire (30); and securely anchoring or embedding the wire (30) in the outer surface (28) of the tube main body (24).

Item 8. The working channel tube (18) according to item 7, wherein the tube main body (24) has an inner diameter between 2 mm and 4.5 mm.

Item 9. The working channel tube (18) according to item 7 or 8, wherein a pitch of the coil (32) is between 0.5 mm and 1.5 mm, especially preferred around 1 mm.

Item 10. The working channel tube (18) according to any one of the items 7 to 9, wherein the elongated hollow tube main body (24) comprises an outer layer (46) comprising the outer surface (28) and an inner layer (48) comprising the inner surface (26), wherein a material of the outer layer (46) is different from a material of the inner layer (48).

Item 11. The working channel tube (18) according to item 10, wherein the materials of the outer layer (46) and the inner layer (48) are both meltable plastic materials and a melting temperature of the material of the inner layer (48) is higher than a melting temperature of the material of the outer layer (46).

Item 12. The working channel tube (18) according to any one of the items 7 to 11, wherein a wall thickness of the elongated hollow tube main body (24) is at least two times greater than a diameter or thickness of the wire (30).

Item 13. Use of a working channel tube (18) according to any one of items 7 to 12 as part of an internal or external working channel (14) of an endoscope (2).

Item 14. An endoscope (2) comprising: an endoscope handle (4) or interface comprising a handle or interface housing and a working channel access port (16); an insertion cord (6) configured to be inserted into a patient's body cavity and comprising an insertion tube (8), an actively bendable bending section (10) and a distal tip unit (12); a working channel (14) extending from the working channel access port (16) of the endoscope handle (4) or interface to the distal tip unit (12) of the insertion cord (6) and comprising the working channel tube (18) according to any one of items 7 to 12.

Item 15. The endoscope (2) according to item 14, wherein the working channel tube (18) extends through the bending section (10) and is locally reinforced by the coil (32) in the bending section (10).

Item 16. A method for producing a reinforced working channel tube for an endoscope, the method comprising: providing a tube main body having a longitudinal axis, the tube main body being an elongate hollow tube having an outer surface comprising a meltable plastic material; providing a wire; heating a portion of the wire; winding the heated portion of the wire under tension around the outer surface of at least a portion of the tube main body to form a coil having a coil pitch, the heated portion of the wire softening or melting the outer surface and penetrating through the softened or melted outer surface of the tube main body; and allowing the softened or melted outer surface of the tube main body to cool, wherein after cooling the coil is affixed to the tube main body.

Item 17. The method of item 16, wherein the tube main body comprises an inner layer and an outer layer, and wherein the inner layer and the outer layer comprise different materials.

Item 18. The method of items 16 or 17, wherein the inner layer has a higher Vicat Softening Point than the outer layer.

Item 19. The method of item 18, wherein the tube main body further comprises a tie layer between the inner layer and the outer layer.

Item 20. The method of any one of items 16 to 19, wherein the coil is at least partially embedded in the outer layer.

Item 21. The method of any one of items 16 to 20, wherein winding the heated portion of the wire comprises: turning the tube main body around its longitudinal axis at a rotational speed; and axially translating the wire along the longitudinal axis of the tube main body at a translational speed, wherein the translational speed and the rotational speed set the coil pitch.

Item 22. The method of any one of items 16 to 21, wherein coil pitch is between 0.5 mm and 1.5 mm.

Item 23. The method of item 22, wherein coil pitch is between 0.8 mm and 1.2 mm.

Item 24. The method of any one of items 16 to 23, wherein heating the portion of the wire comprises passing the wire over a heating element.

Item 25. The method of any one of items 16 to 24, wherein winding the heated portion of the wire under tension around the outer surface of the at least the portion of the tube main body comprises positioning the heated portion of the wire onto the outer surface before a temperature of the heated portion of the wire is lower than a softening point of the meltable plastic material.

Item 26. The method of any one of items 16 to 25, wherein winding the heated portion of the wire under tension comprises tensioning the wire to between 0.5 N and 3.0 N during the winding.

Item 27. The method of item 26, wherein winding the heated portion of the wire under tension comprises tensioning the wire to between 1.6 N and 2.4 N during the winding.

Item 28. The method of any one of items 16 to 27, wherein the tube main body comprises an inner diameter of between 2.0 mm and 4.5 mm.

Item 29. The method of any one of items 16 to 28, wherein the tube main body comprises a wall having a wall thickness of less than 0.5 mm.

Item 30. The method of any one of items 16 to 29, wherein the wall thickness is at least two times greater than a diameter or thickness of the wire.

Item 31. A working channel tube comprising: a tube main body having a longitudinal axis, the tube main body being an elongate hollow tube having an outer surface comprising a meltable plastic material; and a wire wound around the outer surface of at least a portion of the tube main body to form a coil having a coil pitch, wherein the wire is affixed to the tube main body by the method of any one of items 16 to 30 to form the coil.

Item 32. The working channel tube of item 31, wherein the tube main body has an inner diameter of between 2 mm and 4.5 mm.

Item 33. The working channel tube of any one of items 31 to 32, wherein the coil pitch is between 0.5 mm and 1.5 mm.

Item 34. The working channel tube of any one of items 31 to 33, wherein the tube main body comprises an outer layer having an outer surface and an inner layer having an inner surface, wherein a material of the outer layer is different from a material of the inner layer.

Item 35. The working channel tube of any one of items 31 to 34, wherein the material of the inner layer has a Vicat Softening Point higher than a Vicat Softening Point of the material of the outer layer.

Item 36. The working channel tube of any one of items 31 to 35, wherein a wall thickness of a wall of the tube main body is at least two times greater than a diameter or thickness of the wire.

Item 37. An endoscope comprising: an endoscope handle or interface comprising a handle or interface housing and a working channel access port; an insertion cord configured to be inserted into a patient's body cavity and comprising an insertion tube, a bending section and a distal tip unit; and a working channel extending from the working channel access port of the endoscope handle or interface to the distal tip unit of the insertion cord and comprising the working channel tube of any one of items 31 to 36.

### LIST OF REFERENCE SIGNS

2 endoscope

4 endoscope handle

6 insertion cord

8 insertion tube

10 bending section

12 distal tip unit

14 working channel

16 working channel access port

18 working channel tube

20 first operating unit

22 second operating unit

24 tube main body

25 tube main body wall

26 inner surface

28 outer surface

30 wire

32 coil

34 winding apparatus

36 control unit

38 spindle

40 slide

41 slide slot

42 winch

43 slide channel guide surface

44 heating element

46 outer layer

48 inner layer

50 primer

60 segment

62 hinge

64 steering cable

66 steering cable guide

68 wires

70 spacing

72 cut-out lobe

100 visualization system

102 video processing apparatus

104 display screen

106 cable connector

T1 wall thickness

T2 inner layer thickness

T3 outer layer thickness

We claim:

1. A method for producing a reinforced working channel tube for an endoscope, the method comprising:
providing a tube main body having a longitudinal axis, the tube main body being an elongate hollow tube having an outer surface comprising a meltable plastic material;
providing a wire;
heating a portion of the wire;
winding the heated portion of the wire under tension around the outer surface of at least a portion of the tube main body to form a coil having a coil pitch, the heated portion of the wire softening or melting the outer surface and penetrating through the softened or melted outer surface of the tube main body; and
allowing the softened or melted outer surface of the tube main body to cool,
wherein after cooling the coil is affixed to the tube main body.

2. The method of claim 1, wherein the tube main body comprises an inner layer and an outer layer, and wherein the inner layer and the outer layer comprise different materials.

3. The method of claim 2, wherein the inner layer has a higher Vicat Softening Point than the outer layer.

4. The method of claim 3, wherein the tube main body further comprises a tie layer between the inner layer and the outer layer.

5. The method of claim 1, wherein the coil is at least partially embedded in the outer layer.

6. The method of claim 1, wherein winding the heated portion of the wire comprises:
turning the tube main body around its longitudinal axis at a rotational speed; and
axially translating the wire along the longitudinal axis of the tube main body at a translational speed, wherein the translational speed and the rotational speed set the coil pitch.

7. The method of claim 6, wherein coil pitch is between 0.5 mm and 1.5 mm.

8. The method of claim 7, wherein coil pitch is between 0.8 mm and 1.2 mm.

9. The method of claim 1, wherein heating the portion of the wire comprises passing the wire over a heating element.

10. The method of claim 8, wherein winding the heated portion of the wire under tension around the outer surface of the at least the portion of the tube main body comprises positioning the heated portion of the wire onto the outer surface before a temperature of the heated portion of the wire is lower than a softening point of the meltable plastic material.

11. The method of claim 1, wherein winding the heated portion of the wire under tension comprises tensioning the wire to between 0.5 N and 3.0 N during the winding.

12. The method of claim 11, wherein winding the heated portion of the wire under tension comprises tensioning the wire to between 1.6 N and 2.4 N during the winding.

13. The method of claim 1, wherein the tube main body comprises an inner diameter of between 2.0 mm and 4.5 mm.

14. The method of claim 13, wherein the tube main body comprises a wall having a wall thickness of less than 0.5 mm.

15. The method of claim 14, wherein the wall thickness is at least two times greater than a diameter or thickness of the wire.

16. A working channel tube comprising:
a tube main body having a longitudinal axis, the tube main body being an elongate hollow tube having an outer surface comprising a meltable plastic material; and
a wire wound around the outer surface of at least a portion of the tube main body to form a coil having a coil pitch,
wherein the wire is affixed to the tube main body by the method of claim 1 to form the coil.

17. The working channel tube of claim 16, wherein the tube main body has an inner diameter of between 2 mm and 4.5 mm.

18. The working channel tube of claim 16, wherein the coil pitch is between 0.5 mm and 1.5 mm.

19. The working channel tube of claim 16, wherein the tube main body comprises an outer layer having an outer surface and an inner layer having an inner surface, wherein a material of the outer layer is different from a material of the inner layer.

20. The working channel tube of claim 19, wherein the material of the inner layer has a Vicat Softening Point higher than a Vicat Softening Point of the material of the outer layer.

21. The working channel tube of claim 20, wherein a wall thickness of a wall of the tube main body is at least two times greater than a diameter or thickness of the wire.

22. An endoscope comprising:
an endoscope handle or interface comprising a handle or interface housing and a working channel access port;
an insertion cord configured to be inserted into a patient's body cavity and comprising an insertion tube, a bending section and a distal tip unit; and
a working channel extending from the working channel access port of the endoscope handle or interface to the distal tip unit of the insertion cord and comprising the working channel tube of claim 16.
